(19) United States
(12) Patent Application Publication
(10) Pub. No.: US 2019/0265168 A1
(43) Pub. Date: Aug. 29, 2019

(54) MATERIALS AND METHODS FOR THE QUANTIFICATION OF A FLUID

(71) Applicant: MC10, Inc., Lexington, MA (US)

(72) Inventors: Gilbert Lee Huppert, Stoneham, MA (US); Hakan Mutlu, North Chelmsford, MA (US); Brian Murphy, Medford, MA (US)

(21) Appl. No.: 16/346,588

(22) PCT Filed: Nov. 1, 2017
(86) PCT No.: PCT/US2017/059457

§ 371 Date: May 1, 2019

Related U.S. Application Data
(60) Provisional application No. 62/415,861, filed on Nov. 1, 2016.

(51) Int. Cl. G01N 21/78; G01N 33/52; G01N 33/84; G01N 1/28

(52) U.S. Cl. CPC G01N 21/78; G01N 33/523; G01N 2001/362; G01N 1/2813; G01N 33/84

(57) ABSTRACT

A composition for application to a substrate to form a film or deposit for quantifying a fluid, or one or more analytes within a fluid, is disclosed. The composition includes a carrier configured as a transport medium for application of the composition to a substrate and at least one salt of chloranilic acid. The carrier can include at least one compound soluble within a bodily fluid and configured to at least partially solidify upon application of the composition on the substrate. The carrier can optionally include a liquid medium as a bulk transport medium for application of the composition. In some aspects, the liquid medium can be water, the at least one compound can be one or more water-soluble polymers, and the salt of chloranilic acid can be silver chloranilate. A color change in response to the film or deposit dissolving in the fluid and the at least one salt of chloranilic acid reacting with the one or more analytes allows for the quantification of the one or more analytes, and/or a quantification of the volume and/or flow rate of the fluid.

### CROSS-REFERENCE TO RELATED APPLICATIONS

The present application claims the benefit of and priority to U.S. Provisional Patent Application No. 62/415,861, filed Nov. 1, 2016, entitled, “MATERIALS AND METHODS FOR THE QUANTIFICATION OF A FLUID,” which is hereby incorporated by reference herein in its entirety.

### FIELD OF THE INVENTION

The present disclosure relates to the quantification of analytes in a fluid. In particular, the present disclosure relates to the quantification of one or more analytes in a fluid, such as a bodily fluid.

### BACKGROUND OF THE INVENTION

It can be desirable to measure one or more analytes in a fluid, particularly in a bodily fluid. One such analyte in a bodily fluid includes chloride ions in sweat. A measurement of one or more analytes in a bodily fluid should involve non-toxic and biocompatible components given the nature of the fluid being measured, particularly for on-body measurement applications.

Silver chloranilate is a chemical known to react with chloride ions in fluids. However, silver chloranilate can be difficult to use as it exists in a crystalline powder form. Typically, the silver chloranilate powder or solution is added in a solution containing the analytes. These powder and in-solution forms are not useful for on-body detection of chloride ions in a bodily fluid, such as sweat.

Therefore, there is a continuing need for developing materials and methods that solve the above and related problems.

### SUMMARY OF THE INVENTION

Aspects of the present disclosure include a composition in the form of a dispensable liquid, such as an ink (e.g., can be printed), a film, or a deposit (e.g., having a specific shape, volume, and/or weight), that can be applied to a substrate. The composition includes a carrier configured as a transport medium for application of the composition to a substrate and at least one salt of chloranilic acid. In some aspects, the carrier can comprise a liquid medium, which can be water, an alcohol, an oil, a volatile organic compound (VOC), or a combination thereof. In some aspects, the carrier can comprise at least one compound soluble within the bodily fluid and configured to at least partially solidify upon application of the composition on the substrate. In some aspects, the at least one compound can be combined with the liquid medium or the carrier can be the at least one compound alone (i.e., without a separate liquid medium). In some aspects, the at least one compound can be at least one polymer that is soluble in a bodily fluid that is being analyzed. According to some aspects, the at least one polymer can be polyethylene glycol (PEG), polyvinylpyrrolidone (PVP), polyvinyl alcohol (PVA), or a combination thereof. In some aspects, the at least one salt of chloranilic acid can be silver chloranilate.

Additional aspects of the present disclosure include a composition soluble in a fluid, such as a bodily fluid. The composition can be formed of a carrier matrix that is soluble in the bodily fluid, and at least one salt of chloranilic acid that is contained within the carrier matrix. In some aspects, the at least one carrier matrix can be formed of PEG, polyvinylpyrrolidone (PVP), polyvinyl alcohol (PVA), or a combination thereof. In some aspects, the at least one salt of chloranilic acid can be a silver chloranilate.

Still additional aspects of the present disclosure include a method of forming a dispensable composition that is soluble in a fluid, such as a bodily fluid. The method includes forming a composition comprising a carrier configured as a transport medium for application of the composition to a substrate and at least one salt of chloranilic acid. The method further includes applying (e.g., dispensing, printing, depositing, spraying, etc.) the composition on to the substrate, and allowing the carrier to harden resulting in the formation of a film or deposit. According to some aspects, the composition can be about 72 parts by weight of a liquid medium, about 10 parts by weight of at least one polymer, and about 18 parts by weight of at least one salt of chloranilic acid.

Further aspects of the present disclosure include a method of quantifying one or more analytes in a fluid. The method includes contacting a film or deposit with the fluid to cause the film or deposit to at least partially dissolve. The film or deposit includes a carrier matrix that is dissolvable within the fluid and at least one salt of chloranilic acid contained within the carrier matrix. The one or more analytes in the fluid react with the salt of chloranilic acid upon contact to form chloranilic acid. The method further includes determining a concentration of the one or more analytes based on a color (e.g., a color change or lack of color change) of the fluid in response to the formation of the chloranilic acid.

Additional aspects of the present disclosure include a method of quantifying a fluid. The method includes directing a fluid into a channel, the channel having a known volume and being covered, at least in part, with a film or a deposit. The film or deposit includes a carrier matrix dissolvable within the fluid and at least one salt of chloranilic acid contained within the carrier matrix. The at least one salt of chloranilic acid reacts to form chloranilic acid upon contact with one or more analytes in the fluid and the reaction results in a change in color of the fluid. The method further includes determining an extent of fluid flow into the channel based on a measurement of a color front within the channel. The color front is formed by a color change in the fluid in response to the presence of the chloranilic acid as fluid flows over the film or deposit as it travels along the channel. The method further includes determining a volume, a flow rate, or a combination thereof of the fluid based on the extent of fluid flow.

Additional aspects of the disclosure will be apparent to those of ordinary skill in the art in view of the detailed description of various embodiments, which is made with reference to the accompany drawings, a brief description of which is provided below.

### BRIEF DESCRIPTION OF THE DRAWINGS

The disclosure will be better understood from the following description of exemplary embodiments together with reference to the accompanying drawings.

FIG. 1 shows a flowchart of a process for forming a film or deposit, in accord with aspects of the present disclosure.

FIG. 2 shows a flowchart of a process for quantifying the amount of one or more analytes in a fluid, in accord with aspects of the present disclosure.

FIG. 3 shows a flowchart of a process for quantifying the volume and/or flow rate of a fluid, in accord with aspects of the present disclosure.

### DETAILED DESCRIPTION OF THE ILLUSTRATED EMBODIMENTS

Although the present disclosure contains certain exemplary embodiments, it will be understood that the disclosure is not limited to those particular embodiments. On the contrary, the present disclosure is intended to cover all alternatives, modifications, and equivalent arrangements as may be included within the spirit and scope of the disclosure as further defined by the appended claims.

The present disclosure describes a dispensable composition, such as a suspension, a film or deposit, and a method for forming the film or deposit, in addition to methods for analyzing a fluid and/or one or more analytes in a fluid, both quantitatively and qualitatively. The dispensable composition disclosed herein can be dispensed, printed, sprayed, etc. on a substrate and used to form a film or deposit on the substrate. The resulting film or deposit includes a reagent used to analyze the analyte mixed with the fluid soluble carrier matrix having a substantially uniform thickness over a portion of the substrate. The resulting deposit includes the reagent mixed with the fluid soluble carrier matrix having a predefined shape, volume and/or weight (e.g., in the form of a blob, a dot, or other regular or irregular shape). Typically, the dispensable composition is dried on the substrate and remains in the form of a solid or a semi-solid (e.g., a gel), until it interacts with the fluid (e.g., bodily fluid) containing the analyte. The dispensable composition allows for the quantification of one or more analytes within a fluid, such as a bodily fluid, along with properties of the fluid itself (e.g., volume, flow rate, etc.) when the fluid contacts the film or deposit. Quantification of the one or more analytes and/or the fluid can be achieved colorimetrically based on a change in the color of the fluid responsive to the contact of the fluid to the dispensable composition. More specifically, quantification can be achieved by the one or more analytes in the fluid contacting one or more components within the dispensable composition. The flexibility of the substrate (containing the dispensable composition) and the colorimetric analysis of the fluid based on the contact between a component within the dispensable composition and one or more analytes in the fluid allows for the dispensable composition to be applied to on-body patches and similar wearable devices for the on-body quantification of one or more analytes, such as one or more analytes within a bodily fluid, as well as the bodily fluid itself.

Although aspects of the present disclosure are primarily described with respect to sweat as the bodily fluid and chloride ions as the one or more analytes within the bodily fluid, one of ordinary skill in the art would readily appreciate that the present disclosure is equally applicable to other bodily fluids (e.g., tears, bile, breast milk, etc.), or other fluids (e.g., non-bodily fluids), and one or more analytes within the other bodily fluids or non-bodily fluids.

The composition (or suspension) of the present disclosure includes a carrier soluble within a fluid, such as a bodily fluid, and at least one salt of chloranilic acid. In some aspects, the carrier can include a liquid medium that primarily serves as a bulk transport medium for the application (e.g., dispensing, printing, depositing, spraying, etc.) of the at least one salt of chloranilic acid, or the at least one salt of chloranilic acid and at least one compound (e.g., one or more salts, sugars, polymers, and the like, described below), onto a desired substrate. In some aspects, the liquid medium can be selected so that the at least one compound is soluble in the liquid medium. However, in some aspects, the liquid medium can be selected such that the at least one compound is not soluble in the liquid medium, such as in the case of a heterogeneous suspension, where application of the suspension still results in a carrier matrix, as described below.

In some aspects, the at least one compound itself can serve as the bulk transport medium for the application of the at least one salt of chloranilic acid. The at least one compound initially can be in a liquid phase upon combining with the salt of the chloranilic acid and, after application to a substrate, can cool, vaporize, or otherwise harden to form a solid or semi-solid film or deposit containing the at least one salt of chloranilic acid. For example, the carrier can include a polymer in liquid phase that, when applied to the substrate, changes phase to a solid or a semi-solid and forms a carrier matrix that contains the salt of chloranilic acid, as further described below.

Although the composition is described herein primarily as a suspension, the present disclosure is applicable to other types of fluid systems containing one or more compounds (e.g., one or more salts, sugars, polymers, and the like) and one or more salts of chloranilic acid. Such other types of fluid systems include, for example, dispersions, colloids, solutions, mixtures, admixtures, and the like. Thus, although described primarily with respect to a suspension, one of ordinary skill in the art would readily understand that aspects of the present disclosure apply to other types of fluid systems.

In the case where the carrier of the suspension includes a liquid medium, the liquid medium can be, for example, water, an alcohol, an oil, a volatile organic compound (VOC), or a combination thereof. Such alcohols can include, for example, methanol, ethanol, n-propyl alcohol, isopropyl alcohol, and the like, and combinations thereof. In some aspects, a less volatile alcohol can be used (or water can be used) to control (e.g., extend) the working life of the suspension. Extending the working life of the suspension provides additional time between the preparation of the suspension and the application of the suspension to the substrate prior to the liquid medium of the suspension evaporating, ending the working life of the suspension.

As discussed above, the suspension can include at least one compound, either in addition to the liquid medium or without the liquid medium (e.g., the at least one compound is the transport medium). The at least one compound can be selected to be soluble in the fluid being analyzed or containing the analyte (e.g., a bodily fluid). The at least one compound also can be selected so as to form a solid or a semi-solid upon application to the substrate and can dissolve upon contact with the fluid to release the contained salt of chloranilic acid.

In some aspects, the at least one compound can be at least one polymer. The at least one polymer is selected to create a dispensable composition that retains the at least one salt of chloranilic acid after application of the suspension on a substrate and evaporation of the liquid medium, or upon hardening of the at least one polymer (e.g., where the suspension does not include a liquid medium). The at least one polymer also is selected to be soluble in the fluid or bodily fluid that contains the analyte. The solubility of the at least one polymer allows for the release of the at least one salt of chloranilic acid upon contact with the bodily fluid. For example, upon the bodily fluid contacting the at least one polymer, the at least one polymer dissolves, releasing the at least one salt of chloranilic acid. Such polymers for the at least one polymer include water-soluble acrylate polymers, water-soluble cellulose and cellulose derivative polymers, one or more polymeric carbohydrates, including amylopectin, glycogen, cellulose, and other polysaccharides, and the like.

In some aspects, the at least one polymer can be any water-soluble polymer, such as PEG, which can provide for a dispensable composition and resulting deposit with increased flexibility. The increased flexibility is beneficial for applications of the dispensable composition in on-body measurements of the analyte, such as within or on on-body patches or similar devices. In some aspects, the at least one polymer can be entirely PEG. The PEG can have a molecular weight of about 200 to 8,000,000, such as about 8,000. In some aspects, the molecular weight of PEG can be selected so that the PEG forms a solid when dry—setting a lower molecular weight range of the PEG—and still becomes suspended in the liquid medium—setting a higher molecular weight range of the PEG.

In some aspects, the at least one polymer can further include, or alternatively be, one or more additional water-soluble polymers. Such additional water-soluble polymers can increase the rigidity of the resulting dispensable composition while not making the dispensable composition brittle or unable to adhere to a substrate. The one or more additional water-soluble polymers can include, for example, PVP, PVA, or a combination thereof. For example, the at least one polymer can include PEG mixed with PVP or PVA, or both. Alternatively, the at least one polymer can include only PVP, or only PVA, or only both of PVP and PVA. In the case of PVP, the PVP can have a molecular weight of about 10,000 to about 1,300,000, such as about 360,000. The determination of what molecular weight of PVP can be used can be based on the PVP more quickly dissolving in the liquid medium at the lower end of the molecular weight range and having the at least one polymer swell before dissolving at the higher end of the molecular weight range.

In some aspects, the at least one compound can alternatively be one or more salts, such as sodium sulfate, one or more sugars, such as one or more simple sugars, or one or more other compounds that are soluble within the fluid being analyzed, that form a solid or a semi-solid (e.g., in the form of a blob, a dot, or other regular or irregular shape) after application to a substrate, and that can contain the salt of chloranilic acid within the solid or semi-solid.

In the case of sweat as a bodily fluid, the at least one compound is selected so as to be water soluble given that water is the primary component or solvent of sweat. Where the suspension includes a liquid medium that is not water, such as instead an alcohol, an oil, or a VOC, the at least one polymer can be selected as being soluble in the alcohol, oil, and/or VOC, and, optionally, water as well. The water solubility of the at least one compound allows the at least one compound to dissolve in the presence of sweat (e.g., and other aqueous fluids) to react with the at least one salt of chloranilic acid.

Although described as including a liquid medium and the at least one compound, in some aspects the liquid medium can be omitted, as described above. In this aspect, the suspension can exclude the liquid medium of the water, alcohol, oil, and/or VOC. Instead, the at least one compound can act as the liquid medium. For example, one or more compounds can be used that function also as the liquid medium in terms of being a bulk transport medium. In the case of PEG, as an example, PEG can be readily melted at a relatively low temperature. In a melted state, the PEG can be mixed with the salt of chloranilic acid and kept in the melted state. The melted combination of the PEG and the salt of chloranilic acid can then be applied to a substrate. Instead of drying, the resulting deposit can harden through cooling of the melted PEG and the PEG changing phase back to a solid or a semi-solid state. Other compounds can be used besides PEG, such as other polymers that can be melted at a temperature that allows for mixing with the salt of chloranilic acid and application to a substrate. Thus, although aspects are described herein as including both a liquid medium and at least one compound, in some aspects the at least one compound of the present disclosure can also function as the liquid medium of the suspension.

The salt of chloranilic acid is selected such that the salt is insoluble in the bodily fluid. However, the salt of chloranilic acid also is selected such that the salt reacts with the analyte being measured within the bodily fluid. Moreover, the salt of chloranilic acid is selected so that the reaction of the salt with the analyte causes the color of the bodily fluid to change in the presence of chloranilic acid. This change in color can then be used directly or indirectly to determine the concentration of the analyte and/or chloranilic acid in the fluid. The change in color can be determined colorimetrically with a device, as described further below.

In the case of chloride ions in sweat, the chloride ions react with the salt of chloranilic acid to form chloranilic acid, which is highly colored and changes the color of sweat from substantially clear to colored. The color of the sweat is then directly related to the concentration of the chloranilic acid present in the sweat; with the darker the color corresponding to a higher concentration of the chloranilic acid. Because the chloranilic acid is a reaction product of the salt of chloranilic acid and the chloride ions, the color also is directly related to the concentration of the chloride ions prior to the reaction. Thus, the chloride ion concentration can be qualitatively and/or quantitatively determined based on the color of the sweat, as described further below.

In some aspects, the salt of chloranilic acid can be silver chloranilate. Yet, the salt of chloranilic acid can be any salt that is insoluble in water but reacts with chloride ions in sweat. For on-body applications, however, consideration should be given to select a salt of chloranilic acid that is non-toxic and biocompatible, such as silver chloranilate.

Although the carrier is described primarily herein as the liquid medium and the at least one compound, or optionally just the at least one compound, the carrier can optionally include one or more other components. The one or more components can be added to alter the physical and/or chemical properties of the suspension and/or resulting film or deposit, such as increasing the flexibility of the resulting film or deposit or aiding in the application of the suspension on the substrate. In some aspects, the one or more components can be propylene glycol, ethylene glycol, glycerin, and the like, and a combination thereof to alter the properties of the suspension, such as the viscosity, or the properties of the resulting film or deposit, such as the hardness, tackiness, adhesiveness, etc. The one or more components can also optionally include one or more wetting agents, one or more dispersants, one or more preservatives, and/or one or more thickening agents.

In some aspects, the suspension is formed of about 72 parts by weight of the liquid medium, about 10 parts by weight of the at least one compound, and about 18 parts by weight of the salt of chloranilic acid. However, the amounts of the liquid medium, the at least one compound, and the at least one salt of chloranilic acid within the suspension can vary. In some aspects, the suspension can be formed of about 1 to about 99 parts by weight of the liquid medium, such as about 39.2, about 1 to about 50 parts by weight of the at least one compound, such as about 10.8, and about 1 to about 50 parts by weight of the at least one salt of chloranilic acid, such as about 50. In some aspects, the amounts of the three components can be based on having as little of the at least one compound as possible while still resulting in a solid or semi-solid deposit upon hardening, as much of the at least one salt of chloranilic acid as possible, and enough of the liquid medium to control the viscosity of the suspension to the desired amount, such as for the specific application being used.

For purposes of applying the suspension to a substrate, the suspension should have a viscosity that allows the suspension to be applied to the substrate for the particular mode of application. Where the suspension is screen printed, as an example, the viscosity of the suspension is selected to be able to be applied by the screen-printing. Where the suspension is applied using a syringe, the viscosity of the suspension is selected to be able to syringe dispensed. In some aspects, the viscosity of the suspension is about 1 cP to about 200,000 cP such as about 1000 cP. However, the viscosity can vary from the disclosed range depending on, for example, how the suspension is intended to be applied to the substrate.

To vary the viscosity of the suspension, the amount of liquid medium can be varied. Alternatively, the type of liquid medium and, therefore, the viscosity of the liquid medium can be varied. In addition, or as an alternative, to changing the amount and/or type of the liquid medium to vary the viscosity, the amount and/or type of the at least one compound can be varied. For example, in the case of a polymer as the at least one compound, higher or lower molecular weight polymers can be used, and the amounts used can vary, to vary the viscosity of the suspension. Where multiple polymers are used as the at least one compound, each specific polymer can have varying amounts relative to the other polymers. If the at least one polymer includes PVP and PEG, the suspension can be formed of about 1 to about 25 parts by weight of PVP, such as about 8 parts by weight, and about 0.1 to about 10 parts by weight PEG, such as about 2 parts by weight.

The amount of the salt of chloranilic acid can vary depending on, for example, the particular analyte being investigated, the amount of analyte expected over the course of the analysis, etc. In some aspects, the amount of the salt of chloranilic acid that is used can be, for example, one, two, three, four, five, six, or more times the amount of analyte expected to be present in the sample. In some aspects, the amount of the salt of chloranilic acid is overloaded to ensure that the chloranilic acid is not the limiting reagent. In some aspects, the amount of the salt of chloranilic acid used is based on the range of chloride ions typically found in sweat.

Upon the suspension being applied to the substrate, the liquid medium vaporizes, or the at least one compound hardens (e.g., in the case without a liquid medium per se), leaving behind a film or deposit formed of the at least one compound, in the form of a carrier matrix, and the salt of chloranilic acid contained within the carrier matrix. No special process need be implemented for drying and/or hardening the suspension. Instead, the substrate with the applied suspension thereon can be left in atmospheric conditions during which the liquid medium is allowed to vaporize or the at least one compound is allowed to harden. In some aspects, however, the vaporization and/or hardening can be controlled by placing the substrate with the applied suspension in a controlled environment, such as an oven, a room with controlled temperature, pressure, humidity, etc. In the case of hastening the evaporation, the substrate with the applied suspension can be placed in a controlled environment at, for example, 40° C. However, the temperature, pressure, humidity, etc. can all be varied depending on the desired conditions for vaporization of the liquid medium or hardening of the at least one compound, such as the desired time, rate, etc.

The resulting film or deposit formed can include a carrier matrix formed from the at least one compound with the salt of chloranilic acid contained therein. The film or deposit can be stable at atmospheric conditions (e.g., temperature, pressure, humidity, etc.), can be sufficiently flexible (e.g., not brittle) to be able to conform to a flexible substrate, and can adhere to a substrate on which it is applied. In the case of at least one polymer being the at least one compound, the film or deposit can be about 1 to about 99 weight percent of the polymer matrix, such as about 36 percent by weight, and about 1 to about 99 weight percent of the at least one salt of chloranilic acid, such as about 64 percent by weight. More specifically, the film or deposit can be about 29 weight percent of, for example, the PVP and about 7 weight percent of the PEG. However, the amounts of the components in the film or deposit can vary as described above with respect to the concentrations within the suspension. For example, the carrier matrix can be composed entirely of PEG, of PEG with propylene glycol, or of PVP, PVA, or both, with PEG, propylene glycol, or both, including other combinations of the at least one polymer described above, such as organic cellulose polymer.

The resulting carrier matrix of the film or deposit readily dissolves in sweat. Further, the salt of chloranilic acid does not dissolve in sweat considering that the main component of sweat is water. However, the chloride ions in sweat react with the salt of chloranilic acid to form chloranilic acid. The chloranilic acid is highly colored, which changes the color of the sweat. Based on the change in color, the amount of chloride ions in the sweat can be determined directly from the color (e.g., from the amount or intensity of the color change), as well as the total amount of sweat and perspiration rate indirectly from the color, as described below.

Control solutions containing known amounts of analyte (e.g., chloride ions), chloranilic acid, or both can be used to correlate the color of a fluid based on chloranilic acid being in the fluid to the concentration of the analyte (e.g., chloride ions), chloranilic acid, or both. The correlation of color can then be used to generate a plot and/or an algorithm for inputting a colorimetric value to determine an analyte and/or chloranilic acid concentration. Thus, with the known algorithm and/or plot, the concentration of chloride ions in sweat can be determined colorimetrically based on the known correlations of the control solutions with the known chloride ion concentrations and/or the color change resulting from the chloranilic acid and the known reaction between chloride ions and the salt of chloranilic acid.

In some aspects, the film or deposit can be formed in a channel (or a at least a portion of the channel) on a substrate. In use, such as when the substrate is applied to the body of a user, sweat from the user can enter the channel. Upon the sweat entering the channel and contacting the suspension, the carrier matrix dissolves, releasing the salt of chloranilic acid. The chloride ions within the sweat then react with the salt of chloranilic acid, generating a colored front of sweat moving through the channel based on the chloranilic acid. By knowing the volume of the channel and how far the colored front of sweat traveled into the channel, one can calculate the total volume of sweat. Moreover, given two or more readings of the colored front of sweat over time, one can determine the perspiration rate.

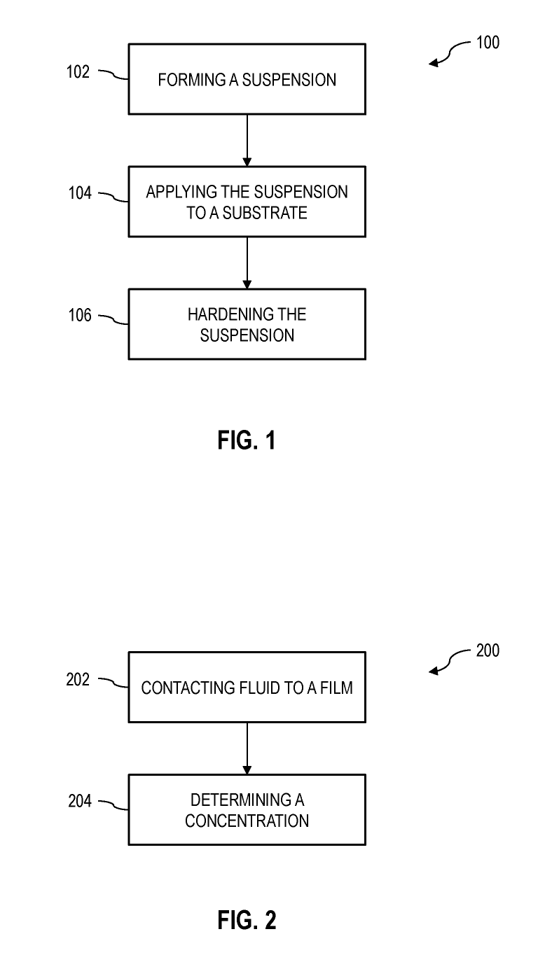

FIG. 1 shows a flowchart of a process 100 for forming a dispensable composition that is soluble in a fluid, such as a bodily fluid, in accord with aspects of the present disclosure. As described above, the film or deposit can be used in the quantification of the fluid and/or one or more analytes in the fluid.

The process 100 begins at step 102 where a suspension (or composition) is formed that includes a carrier soluble in a fluid, such as a bodily fluid, and at least one salt of chloranilic acid. The formed suspension is as described above. For example, the carrier can include a liquid medium and at least one compound soluble within the fluid and configured to at least partially solidify upon loss of the liquid medium after application of the composition on the substrate. Alternatively, the liquid medium can be omitted from the carrier. Moreover, the suspension can be formed according to any process and/or method. In some aspects, and as an example, the at least one compound and the at least one salt of chloranilic acid can be added simultaneously to the liquid medium and mixed. Alternatively, one of the at least one compound or the at least one salt of chloranilic acid can be added to the liquid medium, mixed within the liquid medium, and subsequently followed by the other of the at least one compound or the at least one salt of chloranilic acid. Alternatively, the at least one salt of chloranilic acid can be added to the at least one compound and mixed, followed by the liquid medium being added to the combination of the at least one compound and the salt of chloranilic acid.

At step 104, the suspension is applied to a substrate. Application of the suspension to the substrate can be according to any technique, such as screen-printing, syringe dispensing, spray coating, etc. The suspension can be applied according to a specific pattern or randomly on the substrate. In some aspects, the substrate can include one or more features and the suspension can be applied only on the features. Such features can include, for example, a channel, a ridge, etc.

At step 106, the suspension applied to the substrate is allowed to dry and/or harden such that some or all of the liquid medium evaporates and/or the carrier changes phase (e.g., in the case of the at least one polymer doubling as the liquid medium within the carrier). The drying and/or hardening can occur within normal atmospheric conditions, such as at ambient temperature, pressure, humidity, and the like. In some aspects, the suspension is allowed to dry or harden within a controlled environment, such as an oven, a dehydrator, a clean room, etc. The controlled environment can have controlled temperature, pressure, humidity, etc. to control, for example, the rate of evaporation of the liquid medium to hasten or slow the rate of evaporation as compared to evaporation in ambient conditions. In some aspects, the controlled temperature can be, for example, at 40° C.

The result is a carrier matrix that includes the salt of chloranilic acid contained therein. The carrier matrix further is soluble within a sample that contains, for example, water, alcohol, or both as the main component or solvent. The carrier matrix dissolving releases the salt of chloranilic acid. The released salt of chloranilic can then react with one or more analytes within the sample, such as chloride ions in the case of sweat. The reaction of the salt of chloranilic acid with the chloride ions releases chloranilic acid, which changes the color of the sweat. The change in color of the sweat allows for the concentration of the chloride ions in sweat, along with the sweat volume and the perspiration rate, to be quantified.

FIG. 2 shows a flowchart of a process 200 for quantifying the amount of one or more analytes in a fluid, in accord with aspects of the present disclosure. The process 200 begins at step 202 where a film or deposit is contacted with the fluid. The film or deposit, as described above, is soluble within the fluid. Moreover, the film or deposit is formed of a carrier matrix and at least one salt of chloranilic acid contained within the carrier matrix, as described above.

The fluid containing one or more analytes contacts the film or deposit. In some aspects, the fluid can be sweat and the one or more analytes can be halide ions, such as chloride ions. By contacting the film or deposit with a fluid, at least part of the carrier matrix forming the film or deposit dissolves within the fluid, releasing the at least one salt of chloranilic acid. As described above, the salt reacts with the halide ions to form chloranilic acid, which changes the color of the fluid. In the case of sweat, the substantially clear sweat becomes colored with the formation of the chloranilic acid.

At step 204, a concentration of the one or more analytes is determined based on a color intensity of the fluid in response to the formation of the chloranilic acid. In some aspects, the concentration of the one or more analytes is determined based on colorimetrically analyzing the color of the fluid. In some aspects, the substrate upon which the film or deposit was initially formed can include a device the can analyze the color of the fluid or can be in optical alignment with a device that can analyze the color of the fluid. In some aspects, the device can be a colorimeter, a cell phone, a camera, or another device equipped with a sensor (e.g., a photoresistor, a photocell, a charge-coupled device (CCD), a complementary metal-oxide semiconductor (CMOS), and the like) that can provide a reading, measurement, and/or signal that quantifies the color of the fluid. The device can analyze the absorbance of light at one or more wavelengths. Based on the absorbance, the amount of the analyte, such as chloride ions, can be determined, or the amount of the chloranilic acid can be determined. For example, the absorbance can be compared to previously correlated absorbance of known concentrations of chloride ions in sweat. Alternatively, the absorbance can be inputted into an algorithm that correlates the absorbance to a concentration of the analyte and/or chloranilic acid. In some aspects, the absorbance of light analyzed can be at a wavelength of about 520 nm based on the presence of chloranilic acid to determine the amount of chloranilic acid and/or analyte present in the fluid. However, in some aspects, the color change across the spectrum can be analyzed. In some aspects, the amount of chloranilic acid present can be translated to the amount of chloride based on the 1 to 2 ratio of chloranilic acid to chloride ion, or the amount of chloride can be determined directly based on the known color correlations discussed above.

With the suspension applied to a channel within the substrate, the resulting film or deposit can be used to determine the concentration of one or more analytes within a fluid, as well as one or more qualities of the fluid itself. In some aspects, the qualities can include the volume of the fluid and the flow rate of the fluid.

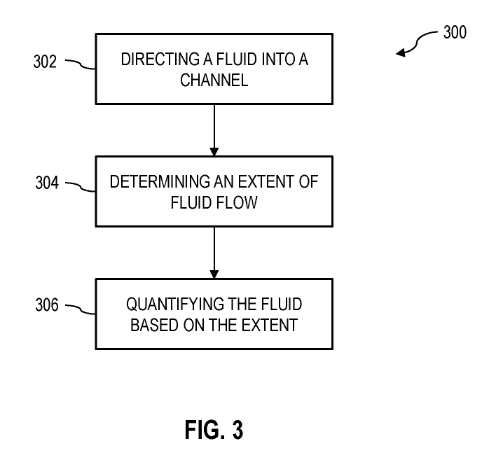

FIG. 3 shows a flowchart of a process 300 for quantifying the volume and/or flow rate of a fluid, such as a bodily fluid, in accord with aspects of the present disclosure. The process 300 begins at step 302 where a fluid is directed into a channel. The channel has a known volume and is covered (e.g., lined, partially filled, etc.), at least in part, with a film or deposit. The film or deposit, as described above, can include a carrier matrix dissolvable within the fluid and at least one salt of chloranilic acid contained within the carrier matrix. As described above, the at least one salt of chloranilic acid reacts to form chloranilic acid upon contact with one or more analytes in the fluid, such as chloride ions where the fluid is sweat.

At step 304, an extent of fluid flow into the channel is determined based on a measurement of a color front within the channel. In the case of sweat as the fluid, the color front is formed by a color change in the sweat as the sweat flows past the film or deposit, and the film or deposit dissolves in the sweat releasing the salt of chloranilic acid. More particularly, the soluble carrier matrix dissolves in the sweat and releases the salt of chloranilic acid, which subsequently reacts with the chloride ions and forms chloranilic acid. The chloranilic acid changes the color of the sweat such that the initial front of sweat passing into the channel is colored rather than substantially clear.

At step 306, a volume, a flow rate, or a combination thereof of the fluid can be determined based on the extent of fluid flow into the channel. In some aspects, the volume can be determined based on the channel having a constant cross-sectional area. The volume of the fluid can be determined by multiplying a length of the extent of fluid flow into the channel by the constant cross-sectional area of the channel.

In some aspects, a flow rate can be determined based on the extent of fluid flow in the channel. In particular, a first extent of fluid flow in the channel can measured at a first time, and a second extent of fluid flow in the channel can be measured at a second time. The flow rate can be determined by calculating a difference between the second extent and the first extent over the period defined by the difference between the second time and the first time. The same procedure can be repeatedly performed to determine changes in the flow rate over time, or an average flow rate over a longer period. In the case of sweat, the flow rate indicates a perspiration rate.

The present disclosure allows for the quantification of one or more analytes in a fluid, as well as quantification of the fluid itself. In the case of sweat as the fluid, the one or more analytes can be chloride ions in the sweat. The determination of the concentration of the chloride ions, as well as the volume of sweat and/or the perspiration rate, can then be used to determine one or more biological parameters of the user for which the analysis was performed. Based on the simplicity of using a color change from the reaction of the salt of chloranilic acid as a way to determine the concentration of the chloride ions and the volume and/or perspiration rate, the present disclosure allows for on-body applications in discrete and discrete patches that, prior to the aspects disclosed herein, were not possible.

Other embodiments are within the scope and spirit of the present disclosure. Further, while the description above refers to the invention, the description may include more than one invention.

1. A composition comprising:
a carrier configured as a transport medium for application of the composition to a substrate; and
at least one salt of chloranilic acid contained within the carrier.

2. The composition of claim 1, wherein the at least one salt of chloranilic acid is silver chloranilate.

3. The composition of claim 1, wherein the carrier comprises a liquid medium of water, an alcohol, an oil, a volatile organic compound, or a combination thereof.

4. (canceled)

5. (canceled)

6. The composition of claim 3, wherein the carrier comprises at least one compound soluble within a bodily fluid and configured to at least partially solidify upon loss of the liquid medium after application of the composition on the substrate.

7. The composition of claim 6, wherein the at least one compound includes at least one polymer of polyethylene glycol, polyvinylpyrrolidone, polyvinyl alcohol, or a combination thereof.

8-18. (canceled)

19. A composition soluble in a bodily fluid comprising:
a carrier matrix soluble in the bodily fluid; and
at least one salt of chloranilic acid contained within the carrier matrix.

20. (canceled)

21. The composition of claim 19, wherein the composition is about 82 parts by weight of the at least one salt of chloranilic acid.

22-28. (canceled)

29. The composition of claim 19, wherein the bodily fluid is sweat.

30. The composition of claim 19, wherein the carrier matrix comprises at least one salt, at least one sugar, or a combination thereof.

31. (canceled)

32. A method of forming a dispensable composition soluble in a bodily fluid comprising:
forming a composition comprising a carrier configured as a transport medium for application of the composition to a substrate and at least one salt of chloranilic acid;
applying the composition to the substrate; and
allowing the carrier to harden resulting in a film or deposit.

33. The method of claim 32, wherein the carrier comprises a liquid medium of water, an alcohol, an oil, a volatile organic compound, or a combination thereof.

34. (canceled)

35. (canceled)

36. The method of claim 33, wherein the carrier comprises at least one compound soluble within a bodily fluid and configured to at least partially solidify upon loss of the liquid medium after application of the composition on the substrate.

37-41. (canceled)

42. The method of claim 32, wherein the carrier is at least one compound soluble within a bodily fluid and configured to be at least partially solid after application of the composition on the substrate.

43. The method of claim 42, wherein the at least one compound is chemically configured to be liquid above room temperature and at least partially solid at about room temperature.

44-46. (canceled)

47. The method of claim 32, wherein the applying of the composition comprises screen printing the composition on the substrate, syringe dispensing the composition on the substrate, or a combination thereof.

48. (canceled)

49. (canceled)

50. The method of claim 32, wherein the carrier is allowed to cool, evaporate, or a combination thereof to at least partially solidify to form a carrier matrix.

51-60. (canceled)
